(19) United States
(12) Patent Application Publication
(10) Pub. No.: US 2005/0059947 A1
(43) Pub. Date: Mar. 17, 2005

(54) Attachable absorbent garment side panels

(76) Inventor: Kathy Murguly, Hillsborough, NJ (US)

Correspondence Address: ROPES & GRAY LLP, ONE INTERNATIONAL PLACE, BOSTON, MA, 02110-2624, US

(21) Appl. No.: 10/942,237

(22) Filed: Sep. 16, 2004

Related U.S. Application Data
(60) Provisional application No. 60/503,532, filed on Sep. 16, 2003.

(51) Int. Cl. A61F 13/15

(52) U.S. Cl. 604/387

(57) ABSTRACT

The present invention discloses an attachable side panel for use with incontinent garments. The side panels are made of a rectangular sheet of skin-friendly material and include a fastener. The fastener is positioned along one or both of the widthwise edges, and allows the side panel to attach to the front and/or back of the incontinent garment bridging the gap on each side of the wearer. The present invention can be used with incontinent garments that are commercially available, or the incontinent garment can be especially designed to be used and sold with the side panels.

### CROSS-REFERENCE TO RELATED APPLICATIONS

This patent application claims the benefit of, and incorporates by reference in its entirety, prior provisional patent application U.S. Provisional Patent Application No. 60/503,532, filed Sep. 16, 2003.

### BACKGROUND OF THE INVENTION

The marketplace provides various size adult absorbent garments (small, medium, large and extra large) for incontinent patients. The extra large size has the largest waist circumference of up to a maximum of 63 to 65 inches. Unfortunately, there are some incontinent patients that require garments which are able to reach a circumference beyond 65 inches. For these individuals, there are no commercially available products that meet their needs.

Manufacturing adult absorbent garments with waist circumferences greater than 65 inches would require specialized, expensive production equipment, capable of handling extremely wide and heavy material rolls. They would also require wider adhesive application systems, wider rotary cutting knives, larger packaging equipment, among other things. When taking all of this into consideration, along with the fact that there are not many incontinent users of this large sized product, one can see why there are no manufacturers of this type of product today. A commercial need exists to provide a product that is a cost effective alternative.

### SUMMARY OF THE INVENTION

The present invention discloses attachable side panels for use with incontinent garments. The side panels are made of a rectangular sheet of skin-friendly material and include a fastener. The fastener is positioned along one or both of the widthwise edges of the side panel and allow the side panel to attach to the front and/or back of the incontinent garment, bridging the gap on each side of the wearer. The present invention can be used with incontinent garments that are commercially available, or the incontinent garment can be especially designed to be used and sold with the side panels.

### BRIEF DESCRIPTION OF THE DRAWINGS

FIG. 1A is a top view of one embodiment of the side panel of the present invention illustrating a substantially parallel sided rectangular shaped panel with the fastener positioned along one edge of the panel for affixing said panel to an absorbent article.

FIG. 1B is a top view of another embodiment of the side panel with the same rectangular shape, but with the fastener positioned intermittently along one edge of the panel.

FIG. 1C is a top view of another embodiment of the side panel of the present invention showing a strip of the fastener positioned along both edges of the panel.

FIG. 2A is a top view of another embodiment of the side panel of the present invention showing a trapezoidal shaped panel with the fastener positioned along one edge of the panel.

FIG. 2B is a top view of a trapezoidal shaped side panel with the fastener positioned intermittently along one edge of the panel.

FIG. 2C is a top view of a trapezoidal shaped side panel with the fastener positioned along both edges of the panel.

FIG. 3 is a perspective view of one embodiment of the side panel of the present invention affixed to a wearer.

FIG. 4 is a top view illustrating two side panels used in combination with a standard absorbent article.

FIG. 5 is a perspective view illustrating two side panels used with a standard absorbent article.

FIG. 6A is a top view of another embodiment of the side panel of the present invention showing a central elastic section positioned between two non-elastic sections. The panel includes a release tape and positioning adhesive.

FIG. 6B is a cross sectional view showing how the sheet of the present invention can be constructed.

FIG. 7A is a perspective view of another embodiment of the invention showing a side panel with folding means positioned in the middle portion of the side panel. The adjustment means can be located on the opposite side of the side panel from the fastener.

FIG. 7B is a top view of the embodiment of FIG. 7A with the adjustment means positioned in the middle portion of the side panel and the fastener positioned along one edge on the opposite side of the side panel.

FIG. 8 is a top view illustrating a three-piece absorbent article with two side panels combined with a rectangular absorbent article.

### DETAILED DESCRIPTION OF THE INVENTION

The present invention provides a means for incontinent bariatric users to use commercially available adult brief products. This result is achieved by providing side panels that attach to each side of any commercially available adult absorbent article, allowing the product to go beyond its widest allowable circumference. It is understood that the side panels can be strung together to expand this distance if necessary.

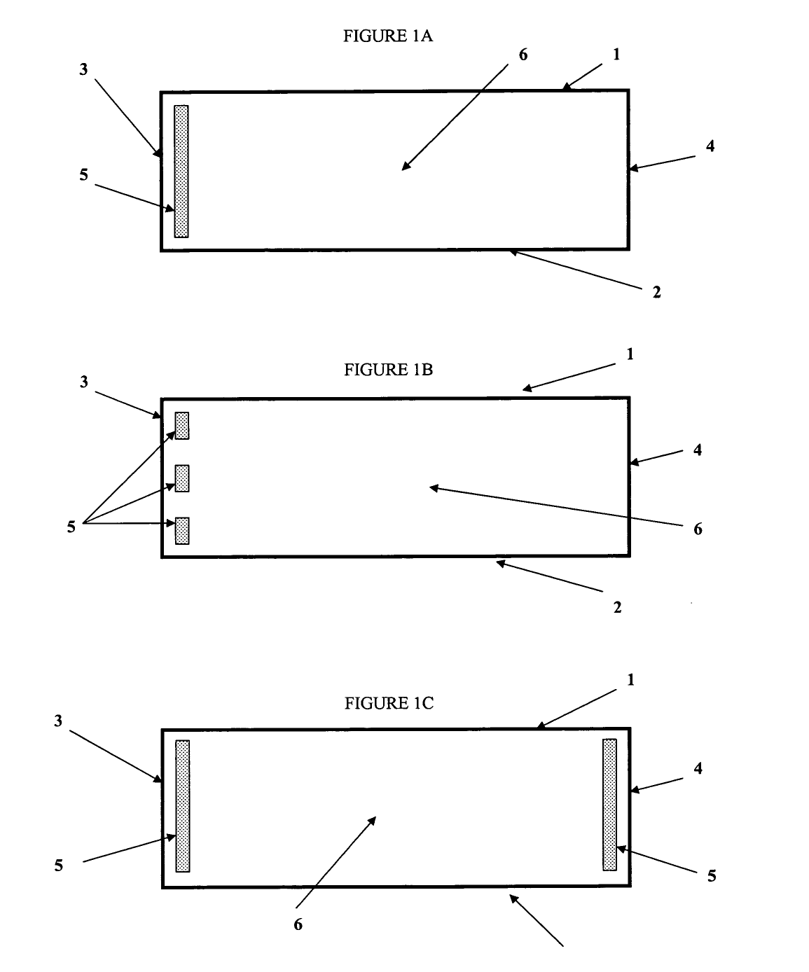

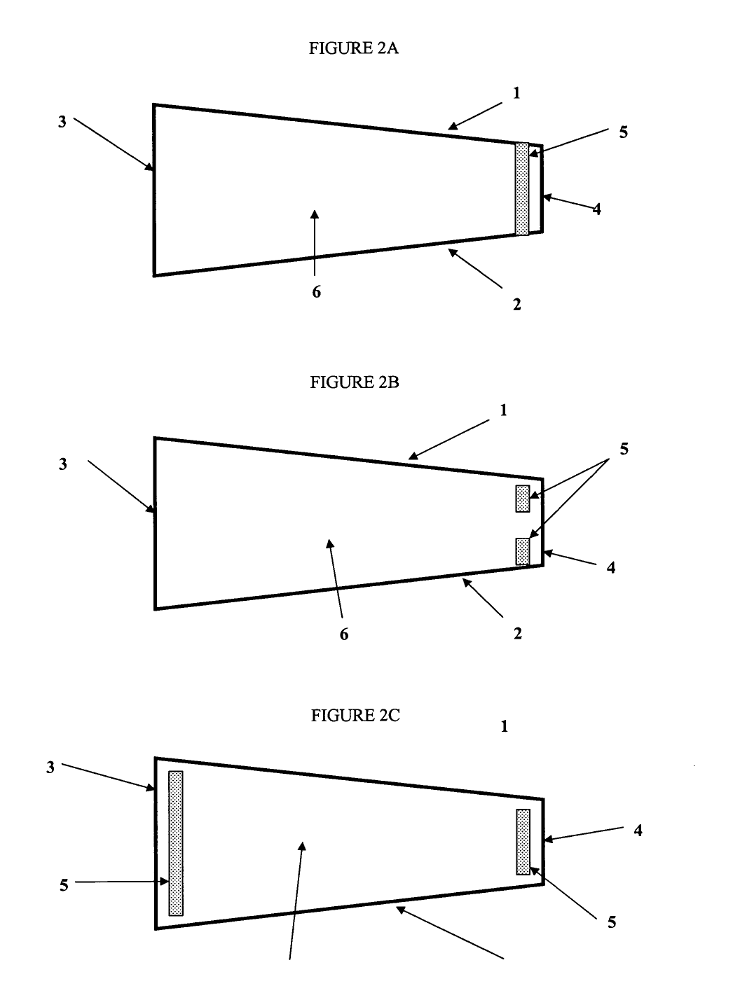

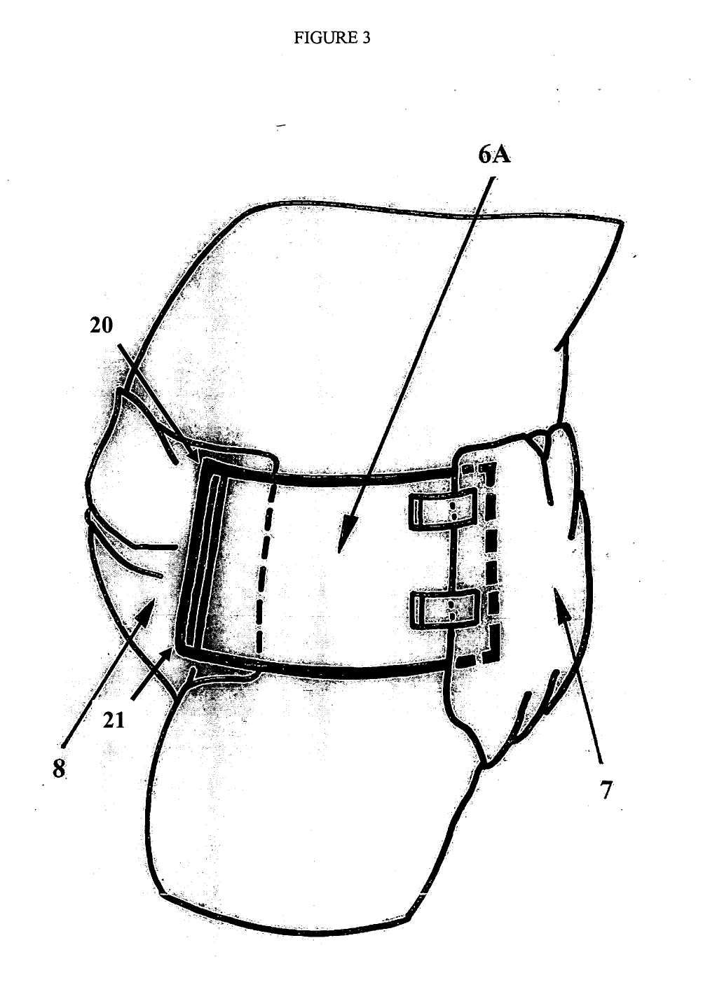

FIGS. 1A-1C and FIGS. 2A-2C illustrate the attachable side panel of the present invention. In these Figures, the side panel comprises a sheet of skin-friendly material 6 having first edge 1 and second edge 2 extending in a lengthwise direction, third edge 3 and fourth edge 4 extending in a widthwise direction. The side panel includes fastener 5 which can be positioned on third edge 3 as shown in FIG. 1A, or which could be positioned on fourth edge 4. The fastener can be applied in a single strip, intermittently or in any other manner as long it allows the side panel to sufficiently attach to the absorbent garment. In the alternative, the fastener can be positioned on both edges of the side panel as shown in FIG. 1C. Whichever way the fastener is applied, it is important that it allow for at least the attachment of upper edge 20 and lower edge 21 of side panel 6A to the upper and lower edges of the front ear of the absorbent garment as illustrated in FIG. 3. In the alternative, the side panel can attach the upper and lower edge of the back ear, if this orientation is so desired.

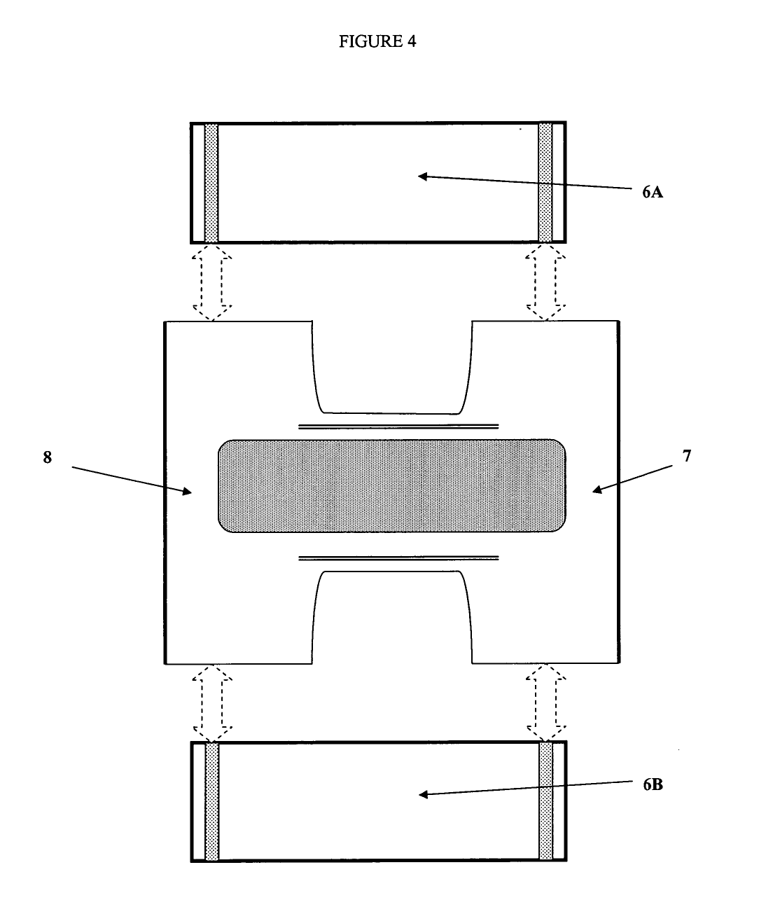

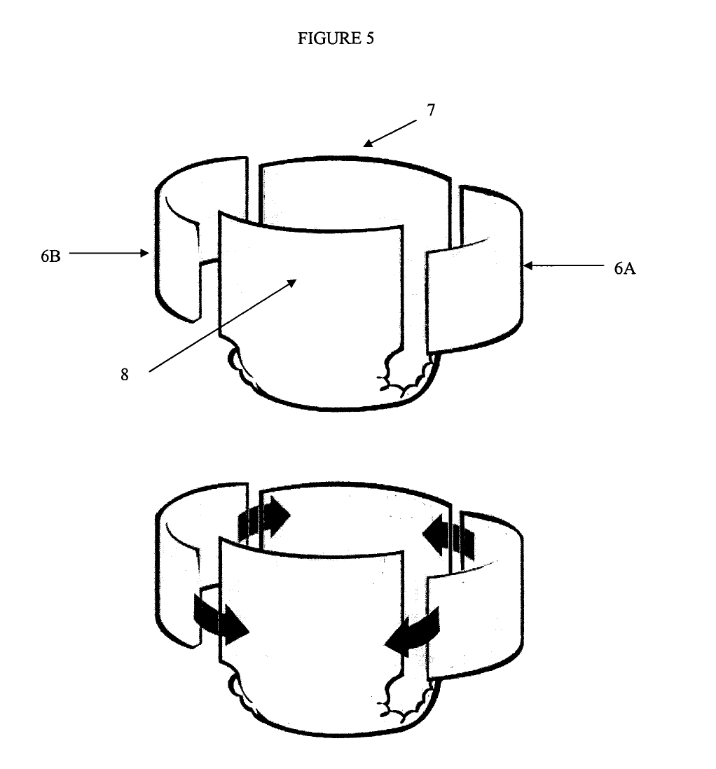

FIG. 4 illustrates how the fastener of one of the side panels is adapted to affix sheet 6A to an absorbent article positioned between the legs of a wearer having a portion positioned in front 8 and back 7 of the wearer, wherein said side panel attaches to the front and back portion of said absorbent article allowing the absorbent article to be secured about one side of the wearer. Second side panel 6B attaches to the absorbent article and secures it about the other side of the wearer. FIG. 5 illustrates the side panels 6A and 6B in a more perspective manner.

The sheet used to make the side panel of the present invention can be made of various skin-friendly materials. The term “skin-friendly”, as used herein, is intended to denote any comfortable material, including breathable material, that will mitigate any discomfort or injury to the wearer when in contact with their skin. One such material is poly laminates commercially available from Clopay Plastic Products of Mason, Ohio. Another acceptable material is a non-woven fabric, such as SBPP (Spundbond Polypropylene) or SMS (Spunbond-Meltblown-Spunbond). Such non-woven materials are commercially available from SAAF Advanced Fabrics of Al-Ahsa, Saudi Arabia. Yet other acceptable materials are other non-woven fabrics such as ABPET (Adhesive Bonded Polyester), TABICO (Through Air Bonded Bi-Component Fiber), TBPP (Thermal Bonded polypropylene), spunlace fibers, hydro-entangled fibers, needle-punched fibers, airlaid composites and woven fabrics. These materials are commercially available from PGI Nonwovens of Mooresville, N.C.

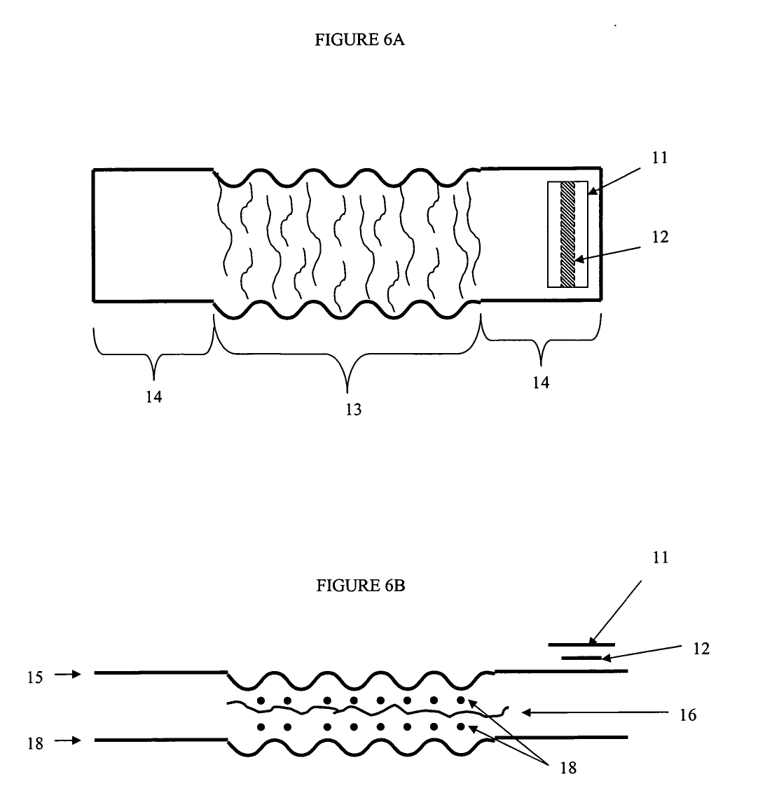

The sheet of the present invention can also be made of an elastic composite, comprising an elastic material sandwiched in-between two layers of non-woven fabric. The elastic composite can also be bonded to one or more non-stretchable materials in sections (not illustrated). Such elastic composites are commercially available from Clopay Plastic Products of Mason, Ohio. In addition, as shown in FIG. 6A, the sheet can have both an elastic section 13 and a non-elastic section 14. FIGS. 6A and 6B also show a fastener comprising release paper 11 and positioning adhesive 12. FIG. 6B, is a cross section of how the elastic sheet in FIG. 6A can be constructed with release paper 11, positioning paper 12, first sheet 15, one or more elastic 16, adhesive 17, and second sheet 18.

In addition, the sheet of the present invention can also be comprised of a breathable poly film and non-woven fabric, which are laminated together. The breathable poly film may be comprised of a micro-porous film, 2-dimensional apertured film, 3-dimensional apertured film, hydro-entangled film, or any combination thereof. Such 3-dimensional apertured poly films and micro-porous films are commercially available from Tredegar Film Products of Richmond, Va.

An acceptable sheet can also be a combination of layers of the materials listed above. Such combinations could be bonded in various ways, such as by adhesive, cohesive, ultrasonic bonding, chemical bonding, heat sealing, poly extrusion, etc.

The sheet of the present invention can include various shapes, such as trapezoidal shapes wherein the third edge is wider in length than the fourth edge or vice versa. In addition, the sheets can be various lengths, but generally have a length greater than its width. Preferably, the width of the sheet is at least about 4 inches and the length of the sheet is at least about 10 inches. Furthermore, the width of the sheet should be sufficient, when attached to the absorbent product, to mitigate slippage of the garment causing it to possibly leak. As mentioned before, it is important that the sheet at least attach at its upper and lower corners in order to sufficiently secure the garment.

The fastener used in the side panel of the present invention can be adapted to affix the side panel by an adhesive such as an adhesive fastening tape. For example, such adhesive fastening tapes are commercially available from 3M Personal Care (3M) of St. Paul Minn. The adhesive fastening tape may be combined with an adhesive release tape, to create a Y-bond attachment configuration. The adhesive release tape is also commercially available from 3M.

In addition, the fastener can be adapted to affix the side panel by using mechanical hooks. One such mechanical hook is commercially available from 3M. Yet another commercially available mechanical hook is Velcro®. The use of mechanical hooks would require a material to attach to such as a loop (brushed or knit), or some other type of engagable material such as a high loft non-woven material. In another embodiment, the adhesive attachable tape and the mechanical attachment system can be combined. Both attachment means can be positioned along side one, another along side two, allowing the user to use either or both. The attachment means can be positioned on either edge or both edges.

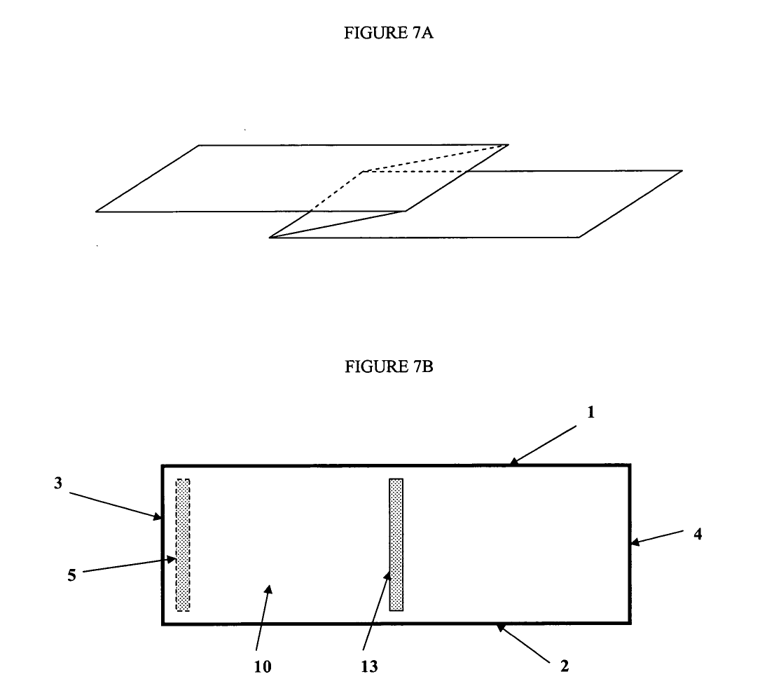

In a further embodiment, the side panel of the present invention can additionally comprise an adjustable means. The term adjustable is intended to include a mechanism that allows the side panel's length to be shortened. As shown in FIG. 7B, this can be accomplished by providing adjustment fastener 13 on the side of the panel opposite the fastener and then folding the side panel in a Z fold as shown in FIG. 7A.

The absorbent article referred to in this patent application can be a standard adult brief product such as DEPENDS®, produced by the Kimberly-Clark Corporation of Dallas, Tex. Other related incontinent products include Belted Undergarments and Refastenable Protective Underwear produced by Kimberly-Clark Corporation of Dallas, Tex. In addition, it may be possible to use the side panels of the present invention with lighter incontinent products such as anatomical Pads (Pad & Pant Systems), produced by SCA Hygiene Products AB of Molndal, Sweden.

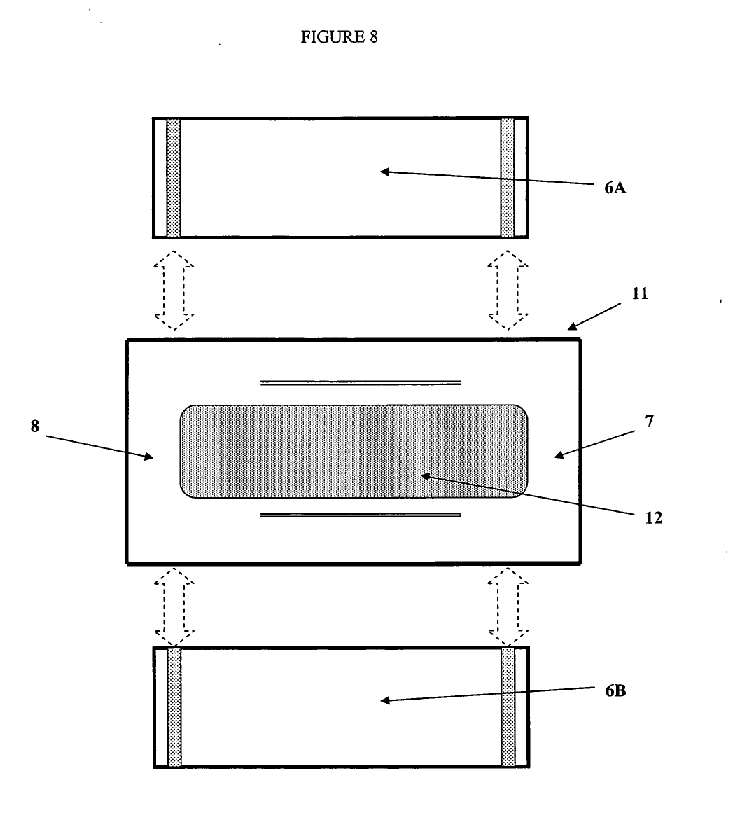

In another embodiment, the present invention can be a three-piece absorbent article as is shown in FIG. 8. To save money, the absorbent piece can be substantially rectangular as shown in FIG. 8, with front portion 8, back portion 7 and absorbent piece or core 12. The basic construction of the absorbent piece is similar to that of a standard adult diaper or a standard light incontinent product. These products generally comprise a liquid permeable top sheet, an absorbent core and a liquid impermeable back sheet. The construction can be similar to a standard adult diaper with leg cut outs, but leg cut outs would be an additional cost to consider. The product would have to be long enough to provide a front and back portion for the side panels to attach to and to be secured about the wearer as shown in FIGS. 3 and 5.

Without further elaboration, the foregoing will so fully illustrate my invention that others may, by applying current or future knowledge, adopt the same for use under various conditions of service.

1. An attachable side panel for use with an absorbent article comprising:
a) a sheet of skin-friendly material, having:
i) a first edge and a second edge extending in a lengthwise direction;
ii) a third edge and a fourth edge extending in a widthwise direction; and
b) a fastener, positioned along the third and/or fourth edge, adapted to affix said sheet to an absorbent article positioned between the legs of a wearer having a portion positioned in the front and back of the wearer,
wherein said side panel attaches to the front and/or back portion of said absorbent article, allowing the absorbent article to be secured about one side of the wearer.

2. The attachable side panel of claim 1, wherein all or a portion of the sheet is elastic.

3. The attachable side panel of claim 1, wherein all or a portion of the sheet is a non-woven material.

4. The attachable side panel of claim 1, wherein the third edge has a width greater than the fourth edge or vice versa.

5. The attachable side panel of claim 1, wherein the sheet's length is greater than its width.

6. The attachable side panel of claim 1, wherein the sheet's length is at least about 10 inches.

7. The attachable side panel of claim 1, wherein the fastener affixes by an adhesive means or by a mechanical means, or combination thereof.

8. The attachable side panel of claim 1, wherein the sheet's width is at least about 4 inches.

9. The attachable side panel of claim 1, wherein the side panel additionally comprises an adjustable means.

10. An attachable side panel for use with an absorbent article comprising:
a) a sheet of skin-friendly material, having:
i) a first edge and a second edge extending in a lengthwise direction;
ii) a third edge and a fourth edge extending in a widthwise direction, wherein the width is less than the length; and
b) a fastener, positioned along the third and/or fourth edge, adapted to affix said sheet to an absorbent article positioned between the legs of a wearer having a portion positioned in the front and back of the wearer,
wherein said side panel attaches to the front and/or back portion of said absorbent article, allowing the absorbent article to be secured about one side of the wearer.

11. The attachable side panel of claim 10, wherein all or a portion of the sheet is elastic.

12. The attachable side panel of claim 10, wherein all or a portion of the sheet is a non-woven material.

13. The attachable side panel of claim 10, wherein the third edge has a width greater than the fourth edge or vice versa.

14. The attachable side panel of claim 10, wherein the sheet's length is at least about 10 inches.

15. The attachable side panel of claim 10, wherein the fastener affixes by an adhesive means or by a mechanical means, or combination thereof.

16. The attachable side panel of claim 10, wherein the sheet's width is at least about 4 inches.

17. The attachable side panel of claim 10, wherein the side panel additionally comprises an adjustable means.

18. A three-piece absorbent article, comprising:
a) two side panels comprising;
i) a sheet of skin-friendly material, having:
a) a first edge and a second edge extending in a lengthwise direction;
b) a third edge and a fourth edge extending in a widthwise direction; and
ii) a fastener, positioned along the third and fourth edges, adapted to affix said sheet to an absorbent article; and
b) an absorbent piece, having:
i) a center portion having an absorbent core that is positioned between the legs of a wearer;
ii) a front portion that is positioned in front of the wearer; and
iii) a back portion positioned in the back of the wearer,
wherein said side panels attach to the front and back portion of said absorbent article securing it about both sides of the wearer.
